(19) United States
(12) Patent Application Publication
(10) Pub. No.: US 2003/0103934 A1
(43) Pub. Date: Jun. 5, 2003

(54) Drugs having long-term retention in target tissue

(75) Inventors: Haruya Sato, Kawasaki-shi (JP); Eiko Hayashi, Kawasaki-shi (JP); Hideyuki Shirae, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 10/259,773

(22) Filed: Sep. 30, 2002

Related U.S. Application Data
(63) Continuation of application No. PCT/JP01/02604, filed on Mar. 28, 2001.

(30) Foreign Application Priority Data
Mar. 30, 2000 (JP) 2000-93775

(51) Int. Cl. A61K 38/19; A61K 38/20; A61K 38/21; A61K 39/395

(52) U.S. Cl. 424/85.1; 424/85.2; 424/85.4; 424/178.1; 514/12; 514/723; 530/351; 530/391.1; 530/399; 568/679

(57) ABSTRACT

The present invention relates to a polyethylene glycol-bound ligand in which polyethylene glycol is bound to a ligand having a binding affinity for a specific receptor or a specific protein (i.e., an antigen), wherein the polyethylene glycol-bound ligand is not internalized into cells, a novel medicament in which a drug is introduced into the polyethylene glycol chain of the ligand, and a pharmaceutical composition containing the same as an effective ingredient

### BACKGROUND OF THE INVENTION

1. Field of the Invention

The present invention relates to a polyethylene glycol-bound ligand in which polyethylene glycol is bound to a ligand having a binding affinity for a specific receptor or a specific protein (i.e., an antigen), wherein the polyethylene glycol-bound ligand is not internalized into cells, a novel medicament in which a drug is introduced into the polyethylene glycol chain of the ligand, and a pharmaceutical composition containing the same as an effective ingredient.

2. Discussion of the Background

In recent years, gene recombination technology and large-scale (mass) cell culture methods have been employed to develop several proteinous medicaments. However, the resulting medicaments have been plagued by prominent defects, such as: (1) the requirement for administration in a high dosage due to a susceptibility of in vivo enzymatic degradation and poor blood-retention; and (2) an increase in occurrence of side effects resulting from poor physiological selectivity, specifically due to the distribution around other organs or cells rather than the desired target.

For example, interferon α (IFNα) or interferon β (IFNβ), an agent for treating chronic hepatitis C, must be administered in a high dose three times a week over a long term to provide an antiviral effect to virally infected hepatic parenchymal cells. However, various additional activities, such as cell growth inhibition, immune response control, MHC antigen expression control and the like, are expressed by the binding of IFNα or IFNβ to IFN receptors on the surface of cells that are normally distributed throughout the body. Accordingly, this prolonged treatment gives rise to side effects such as pyrexia, reduction of platelets or granulocytes, interstitial pneumonia, abnormal thyroid function and the like.

Since these side effects and attending a hospital three times a week greatly influence the patient's quality of life, a long-term retention-type polyethylene glycol-modified IFNα has been developed in recent years as an approach to solve these problems. In this polyethylene glycol-IFNα, polyethylene glycol has been introduced into IFNα, with the result that the half-life in blood has been prolonged and administration once a week has shown the same antiviral effect as the past administration three times a week. However, this strategy has proven problematic since a target orientation is low and the polyethylene glycol-IFNα is retained in the body at a high concentration resulting in an increased incidence of side effects, such as pyrexia.

An alternative strategy has been the development of medicaments of polyethylene glycol modified interleukin-2, since interleukin-2 is expected to be an immunotherapeutic agent of cancers. However, a high degree of toxicity has been associated with this strategy, and as such this strategy has been abandoned.

In general, the past modification of proteinous medicaments (medicines) with polyethylene glycol has advantages, such as dosage reduction and a decreased frequency in dosage administrations arising from the enhanced retention of the drug in the target site.

However, this strategy is problematic in that the drug is also retained in organs other than the desired targets and the side effects flowing therefrom are the same as, or higher than, those of unmodified products.

In order to solve these problems, the present inventors of this application succeeded in using, as a liver recognition element, an artificial ligand (refer to Japanese Patent Kokai Publication JP-A-5-202085) having an appropriate binding affinity to an asialoglycoprotein receptor specifically expressed in hepatic parenchymal cells. In addition, this ligand was not readily incorporated into the cells. Accordingly, this artificial ligand directly bound to interleukin-2, one of proteinous medicaments, to form an artificial ligand-modified proteinous medicines, which could be accumulated in the liver to increase the pharmaceutical effect locally in the liver (refer to WO 98/13381). Although this artificial ligand-modified proteinous medicine enabled the liver accumulation of the physiologically active protein and the expression of its pharmaceutical function through the general administration, it was insufficient with respect to the long-term retention of the drug in the liver and the long-term persistence of the pharmaceutical effect.

Under these circumstances, the present inventors of this application synthesized an artificial ligand and polyethylene glycol-modified proteinous medicament (medicine) with one molecule of an artificial ligand and one molecule of a polyethylene glycol introduced respectively in a fusion protein having a base sequence of a transglutaminase in the N-terminus of interleukin-2. In the artificial ligand and polyethylene glycol-modified proteinous medicine, the modified proteinous medicine having a synergistic effect of the accumulation in the liver and the retention in blood showed that it was present specifically in the liver at a high concentration for a long period of time owing to the synergistic effect of the accumulation in the liver and the retention in blood. However, since the two different elements were bound with separate transglutaminases, this proteinous medicine possessed a tremendous disadvantage in that the production step was intricate and a recovery rate of the final purified product was approximately 1%. Moreover, that final product was not easy to identify and prove the respective binding sites of the two elements and it was difficult to prove constant qualities as a medicine.

Accordingly, there remains a critical need for the development of medicaments that bind to specific receptors, proteins, or antigens on a target cell or tissue, which are easily synthesized, readily retained in vivo, and have minimal side effects.

### SUMMARY OF THE INVENTION

Accordingly, it is an object of the present invention to provide products and methods for retaining drugs in a target tissue using polyethylene glycol that is bound to a ligand, which binds to a specific receptor, protein, or antigen on a target cell or tissue. Such a polyethylene glycol-bound ligand may be conjugated to a drug or a biologically active substance, such as an interleukin or interferon. The effective concentration of such a ligand, and any conjugated drug or biologically active component, is increased at the target cell or target tissue surface, because it selectively binds to and accumulates at the specific target and its removal by internalization or incorporation into the cell or tissue is inhibited. Pharmaceutical compositions, medicaments, and methods of treatment comprising such polyethylene-bound ligands are also disclosed.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows the results of in vivo behavior as described in Example 9, indicating a concentration of accumulation in the liver. In each bar graph the samples (from left to right) are unmodified IFNα, (Gal)₃(6)-IFNα, PEG12-IFNα and (Gal)₃(6)-PEG12-IFNα. FIG. 1a: 3 minutes after administration; FIG. 1b: 60 minutes after administration.

FIG. 2 shows the results of in vivo behavior after intravenous administration to mice as described in Example 9, indicating a concentration in plasma. In each bar graph the samples (from left to right) are unmodified IFNα, (Gal)₃(6)-IFNα, PEG12-IFNα and (Gal)₃(6)-PEG12-IFNα. FIG. 2a: 3 minutes after administration; FIG. 2b: 60 minutes after administration.

### DETAILED DESCRIPTION OF THE INVENTION

Unless specifically defined, all technical and scientific terms used herein have the same meaning as commonly understood by a skilled artisan in polymers and materials chemistry.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based in part on the Inventor's surprising discovery that the foregoing problems may be solved by using, as a ligand for accumulating a drug in a target tissue at a high concentration, a ligand having a binding affinity to a specific receptor or a protein, such as an antigen or the like that is present on a cell membrane of a target tissue. The ligand, according to the present invention, is bound to a polyethylene glycol chain to form a polyethylene glycol-bound ligand, which may also have a drug introduced therein. Also within the context of the present invention, the polyethylene glycol-bound ligand inhibits cellular internalization thereof.

In a preferred object of the present invention is a polyethylene glycol-bound ligand in which a polyethylene glycol chain is bound to a ligand that has a binding affinity to a specific receptor or a protein, such as an antigen or the like, which is present on a cell membrane of a target tissue and has an ability to avoid cellular internalization. Further, the present invention is a medicament containing a polyethylene glycol-bound ligand derivative in which a drug is bound to the polyethylene glycol chain of the polyethylene glycol-bound ligand, or a medical composition containing the medicament as an effective ingredient.

An embodiment of the present invention is a polyethylene glycol-bound ligand in which a polyethylene glycol chain is bound to a ligand having a binding affinity to a specific receptor or a protein such as an antigen or the like present on a cell membrane of a target tissue and, which avoids and/or inhibits cellular internalization and may be used as an active ingredient in a medicament when bound to a drug of which the activity has been characterized.

With respect to the molecular weight of the drug to be used in the medicament of the present invention, it is preferred that the drug that is bound to a polyethylene glycol chain of the polyethylene glycol-bound ligand have a weight-average molecular weight of 60,000 Da or more for elimination in the kidney. However, a weight-average molecular weight of not more than 60,000 Da may also be used. For this purpose, a weight-average molecular weight of polyethylene glycol used in the production may properly be determined from a molecular weight of a drug used and a molecular weight of a ligand used. However, the weight-average molecular weight of the polyethylene glycol is preferably 5 kDa or more.

The target tissue of the present invention is an organ within a living body. It is not particularly limited so long as it becomes a target for accumulating a desired drug at a high concentration for therapy. Examples thereof can include a liver, a kidney, a bone marrow, a pancreas and the like.

The specific receptor or the protein, such as the antigen or the like, present on the cell membrane of the target tissue within the context of the present invention should have an affinity to the ligand in the present invention. However, it is preferable that the specific receptor or the protein present on the cell membrane does not have an affinity to the drug. Further, for formation of the medicament intended by the present invention, the receptor is preferably specific to the target tissue. However, it is not necessarily specific thereto, and it may be a receptor in which a ligand can be accumulated in a target tissue. For example, when the liver is a target, an asialoglycoprotein receptor (ASGP-R) or the like expressed in a large amount in hepatic parenchymal cells is preferable. In addition to this receptor, for example, a mannose receptor quite selectively expressed in hepatic non-parenchymal cells, and an epidermal cell growth factor (EGF) receptor, an insulin receptor, a transferrin receptor, a folate receptor and the like having a low cell specificity in expression but a high receptor density are mentioned.

On the other hand, examples of a protein expressed on a cell membrane except the receptor can include, for example various transporters, antigens and the like.

When a ligand having a binding affinity to compounds is present, the expression of which in specific cancer cells is increased in comparison to normal cells except the specific receptor and the protein and these compounds exist on the target cell membrane, they can also be a subject of the ligand in the present invention.

The ligand in the present invention is not particularly limited so long as it has the binding affinity to the specific receptor or the protein present on the cell membrane of the target tissue and has the ability to avoid and/or inhibit cellular internalization, contains a functional group capable of being bound to the polyethylene glycol chain and does not show side effects in vivo. Moreover, this ligand may be a ligand which acts itself as a drug. However, it is preferable that the ligand itself does not have a physiological activity.

According to the present invention, when the ligand has a binding affinity to a specific receptor present on a cell membrane of a target tissue, a ratio of a dissociation rate constant and an internalization rate constant of the ligand to the specific receptor (a dissociation rate constant of the ligand to the specific receptor/an internalization rate constant of the ligand to the specific receptor) can be 1 or more. Preferably, a certain affinity and a certain ability to avoid internalization are required, that is, a dissociation constant has to be 10 mM or less or an internalization rate constant has to be less than 0.1 min⁻¹.

Such a ligand can be obtained from ligands known to have a binding affinity to a specific receptor or a protein, such as an antigen or the like present on a cell membrane of a target tissue. The ligand may also be obtained: from screening to a desired specific receptor or a desired protein; by screening from the ligands designed on the basis of a structure of a desired specific receptor or a desired protein; or, formed through synthesis by a method according to Japanese Patent Application No.11-186761 or WO01/02851).

Incidentally, when a ligand is artificially synthesized to a protein on a target cell membrane, it is easier to design the ligand for a specific receptor rather than to other expression proteins.

The screening method includes a method which comprises incubating a ligand or a ligand-modified substance having a binding affinity to a specific receptor or a protein such as an antigen or the like (hereinafter referred to as a “receptor or the like”) present on a cell membrane in the presence of free cells or culture cells expressing the receptor. The cells are then washed and a labeled ligand is added to the receptor or the like. The mixture is subsequently incubated at a low temperature to suppress and/or inhibit cellular internalization, followed by screening for a ligand or a ligand-modified substance in which the binding amount of the labeled ligand to the cell surfaces is higher than the binding amount when adding the non-labeled substance of the ligand having the affinity itself.

This latter screening method comprises incubating a ligand or a ligand-modified substance having a binding affinity to a specific receptor or the like present on a cell membrane in the presence of free cells or culture cells expressing the receptor or the like. The cells are then washed with a buffer containing a chelating agent or an acid buffer, and subsequently adding a labeled ligand having an affinity to the receptor or the like. The mixture is then incubated at a low temperature to suppress and/or inhibit cellular internalization and then the mixture is screened for a ligand or a ligand-modified substance in which the binding amount of the labeled ligand to the cell surfaces is larger than the binding amount in adding a ligand in which a ratio of a dissociation rate constant and an internalization rate constant, that is a value of the dissociation rate constant/the internalization rate constant, after binding to the receptor or the like is at least 1. And then this method is repeated until the desired ligand has been identified.

An exemplary ligand to the liver would be a ligand to an asialoglycoprotein receptor expressed specifically in hepatic parenchymal cells and having a branched structure with galactose or N-acetylgalactosamine. A structure used in the branching is preferably a structure branched with an acidic or basic amino acid. However, other compounds capable of making at least biantennarying, such as trishydroxymethylaminomethane and the like, may be used.

Examples of suitable compounds include compounds represented by the following general formula (I).

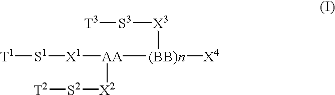

wherein T¹, T² and T³ may be the same or different, and each represents galactose (Gal) or N-acetylgalactosamine (GalNAc),

S¹, S² and S³ may be the same or different, and each represents (CH₂)ₚ or (CH₂CH₂O)_{q} in which p is an integer of 1 to 18 and q is an integer of 1 to 6,

AA and BB may be the same or different, and each represents a basic amino acid or an acidic amino acid,

X¹, X² and X³ may be the same or different, and each represents —CO— when bound to an amino group of an amino acid represented by AA or BB, or —NH— when bound to a carboxyl group of an amino acid represented by AA or BB,

X⁴ represents —COOH when bound to an amino group of an amino acid represented by AA or BB, or —NH₂ when bound to a carboxyl group of an amino acid represented by AA or BB,

n represents 0 or 1, and

when n is 1, amino acids represented by AA and BB may be amide-linked in any positions of the α-positions, the α-position and the γ-position, the γ-position and the α-position and the γ-positions.

Examples of ligands according to formula (I) are described in Japanese Patent Kokai Publication JP-A-202085. Examples of the foregoing basic amino acid and acidic amino acid can include lysine, glutamic acid, aspartic acid and the like.

In the foregoing formula (I), a ligand represented by the following formula (II) is especially preferable.

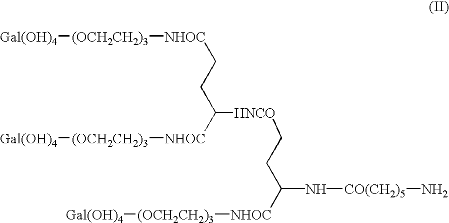

Polyethylene glycol used in the polyethylene glycol chain of the present invention has to have reactive functional groups at both ends in view of qualities of the present invention. The reactive functional groups may be functional groups capable of forming a chemical bonding with the ligand and the drug in the present invention. Examples thereof can include a hydroxyl group, an amino group, a carboxyl group, a vinyl group, an imidazole group, a mercapto group and the like. An alkylene group such as a methylene group, an ethylene group or the like, a sulfonyl group, a carbonyl group, an ether group, a disulfide group or the like may be present between these functional groups and the polyethylene glycol. For example, the following polyethylene glycol derivatives are available:

NH₂—CH₂CH₂—O—(CH₂CH₂O)ₙ—CH₂CH₂—NH₂

NH₂—CH₂—O—(CH₂CH₂O)ₙ—CH₂—NH₂

HOOC—O—(CH₂CH₂O)ₙ—COOH

HOOC—CH₂CH₂—COO—(CH₂CH₂O)ₙ—CO—CH₂CH₂—COOH

HOOC—CH₂—O—(CH₂CH₂O)ₙ—CH₂—COOH

CH₂═CH—SO₂—(CH₂CH₂O)ₙ—CH₂CH₂—SO₂—CH═CH₂

(C₅NH₄)—S—S—(CH₂CH₂O)ₙ—S—S—(C₅NH₄)

HS—(CH₂CH₂O)ₙ—CH₂CH₂—SH

NH₂—CH₂CH₂—O—(CH₂CH₂O)ₙ—COOH

NH₂—CH₂CH₂—O—(CH₂CH₂O)ₙ-CH₂—COOH

NH₂—CH₂CH₂—O—(CH₂CH₂O)ₙ—CO—CH₂CH₂—COOH

NH₂—CH₂—O—(CH₂CH₂O)ₙ—COOH

NH₂—CH₂—O—(CH₂CH₂O)ₙ—CH₂—COOH

NH₂—CH₂—O—(CH₂CH₂O)ₙ—CO—CH₂CH₂—COOH

CH₂═CH—SO₂—(CH₂CH₂O)ₙ—COOH

CH₂═CH—SO₂—(CH₂CH₂O)ₙ—CH₂—COOH

CH₂═CH—SO₂—(CH₂CH₂O)ₙ—CO—CH₂CH₂—COOH

In the foregoing description, n represents an integer, such that the weight-average molecular weight of polyethylene glycol and polyethylene glycol derivatives can range from approximately 1 kDa to approximately 100 kDa. It is more preferable that the average weight-average molecular weight thereof ranges from 3 kDa to 20 kDa and the range of the weight-average molecular weight is as small as possible.

The polyethylene glycol-bound ligand of the present invention can be produced by condensing polyethylene glycol derivatives and a ligand using ordinary techniques of organic synthesis such as an amide linkage, an ester linkage, an ether linkage, a disulfide linkage or the like depending on the types of functional groups thereof.

In a preferred embodiment, the desired polyethylene glycol-bound ligand can be produced by, for example, introducing an alkylamine in which a terminal amino group is protected with a protective group such as Boc group or the like into one end of polyethylene glycol having carboxylic acids (carboxyl groups) in both ends, then introducing a ligand into an opposite end of the polyethylene glycol molecule and removing the protective group for the amino group. When the ligand is introduced into the opposite end of the polyethylene glycol molecule, another functional group may be protected for binding the polyethylene glycol to a desired site of the ligand.

Further, if necessary, it is also possible that a branched chain having two or more functional groups is introduced into one end of the polyethylene glycol and plural ligands are introduced.

The medicament (the polyethylene glycol derivative) in which the drug is bound to the polyethylene glycol chain of the polyethylene glycol-bound ligand in the present invention is one in which the ligand, the polyethylene, and the drug are bound in the order of ligand-polyethylene glycol-drug. For example, groups which become chains for binding them may be introduced therebetween.

In an embodiment of the present invention, the drug in the medicament in which the drug is bound to the polyethylene glycol chain of the polyethylene glycol-bound ligand includes physiologically active proteins such as plasma ingredients, e.g. immunoglobulins, blood coagulation factors and the like, cytokines, e.g. interleukins, interferons α/β/γ, tumor necrosis factor (TNF) and the like, cell growth factors, e.g. a hepatocyte growth factor (HGF), an epidermal cell growth factor and the like, and antioxidases, e.g. superoxide disumtase (SOD), catalase, thioredoxin and the like, and physiologically active peptides such as hormones, e.g. growth hormones, insulin and the like, and immunoreaction control factors, e.g. thymosin α and the like. The origin of these physiologically active proteins or physiologically active peptides is not particularly limited, and they may be derived from animals, plants or microorganisms. Further, proteins expressed and produced by incorporating genes or mutants of these proteins into E. coli, yeasts, Chinese hamster ovary cells or the like are also suitable. Still further, a chimera with other proteins is also available. Moreover, it is preferable that the physiologically active proteins or peptides in the present invention can be purified as much as possible before use to minimize the influence by co-existent proteins.

In another embodiment of the present invetion, the drugs may be drugs of low-molecular compounds having a high toxicity and posing a problem of side effects owing to distribution in sites other than a target site, for example, antitumor agents such as adriamycin, mitomycin C, methotrexate and the like and antiviral agents such as azidothymidine (AZT), adenine arabinoside (ara-A), ribavirin and the like.

The low-molecular compounds having such a high toxicity can also be introduced into the polyethylene glycol-bound ligand of the present invention through natural proteins such as albumin, globulins and the like, various monoclonal antibodies, polysaccharides such as dextran, chitin, chitosan, inulin and the like, polyamino acids such as polylysine, polyglutamic acid and the like, synthetic polymers such as a divinyl ether maleic anhydride copolymer (DIVEMA), a styrene maleic anhydride copolymer (SMA), polyvinyl alcohol and the like, and lipid aggregate carriers such as liposome, lipid microspheres and the like, which can be used as carriers thereof.

The medicament in which the drug is bound to the polyethylene glycol chain of the polyethylene glycol-bound ligand, may be produced by first forming the polyethylene glycol-bound ligand is previously produced and then binding the drug to the polyethylene glycol chain thereof, or by binding the polyethylene glycol to the drug to produce a polyethylene-bound drug and then binding the ligand thereto.

In the present invention, the ligand, polyethylene glycol and the drug can be bound in a usual manner. The binding between them is preferably conducted through a covalent bond, but is not limited thereto. The binding can be conducted by a chemical method or by a method using an enzyme such as a transglutaminase or the like.

Further, the molar ratio of ligand:polyethylene glycol:drug is not necessarily 1:1:1. Within the context of the present invention, it is permissible for the ligand:polyethylene glycol:drug ratio to be 2:1:1, 2:2:1, 3:1:1, 3:3:1 or the like.

A method of binding the drug and the polyethylene glycol chain of the polyethylene glycol-bound ligand or the polyethylene glycol can include the following methods.

First, when the drug is a protein, it is preferable, as a medicine, that the structure thereof is uniform and its biological activity is not decreased. Therefore, it is possible that an alkylamine is introduced into the polyethylene glycol chain or polyethylene glycol of the polyethylene glycol-bound ligand and reacted with the protein using a transglutaminase (refer to WO 96/06181 and WO 96/10089). In view of conducting selective site modification of a specific glutamine residue only, it is especially preferable to use a microbial transglutaminase described in the official gazette of WO 96/10089.

Specifically, a physiologically active protein or peptide, the polyethylene glycol-bound ligand and a transglutaminase, more preferably a microbial transglutaminase are reacted in an aqueous solution. In a preferred method the aqueous solution should have a pH of approximately 7.5 and the reaction should be at room temperature for 12 hours. The concentration ratio of the physiologically active protein or peptide and the polyethylene glycol-bound ligand is preferably in the range of 1:100 to 1:2,000. Further, the amount of the transglutaminase used is 0.01 to 1 unit per 1 nmol of the protein.

As another introduction method, it is also possible to use a method which comprises introducing SPDP (N-Succinimidyl-3-(2-pyridyldithio)propionate), SMPT (Succinimidyloxycarbonyl-α-methyl-α-(2-pyridyldithio)toluene), SMCC (Succinimidyl-4-(N-maleimidomethyl)cyclohexane-1-carboxylate) or the like into the polyethylene glycol chain of the polyethylene glycol-bound ligand or the polyethylene glycol and reacting the protein therewith to selectively modify only a Cys residue in the protein (see Goodson and Katre, 1990, Biotechnology 8, 343-346, and Benhar et al., J. Biol. Chem. 269, 13398-13404).

Moreover, when a protein in which the amino acid in the N-terminus is serine or threonine is used, a method in which an aminoxy derivative is specifically introduced into the amino group in the N-terminus by controlling reaction conditions such as a pH value and the like (refer to Bioconjugate Chem. 1996, 7, 38-44) are preferred.

In addition, the introduction can be also conducted by random modification, for example, a lysine residue side chain amino group modification method using a polyethylene glycol-bound ligand with a functional group introduced, such as a trichloro-s-triazine method, a carboxyimidazole method, a succinimidyl succinate method or the like, which is low in reaction selectivity, though.

Meanwhile, when the drug is a low-molecular compound, various methods such as a cyanogen bromide method, a periodate oxidation method, a carbidiimide method, a glutaraldehyde method, a mixed acid anhydride method, an SPDP reagent method and the like are mentioned. Incidentally, in this case, easy decomposition for exhibiting a pharmaceutical activity within and without cells at good efficiency, namely easy separation of a prodrug-like ligand has also to be taken into consideration.

The binding of these low-molecular compounds and the polyethylene chain of the polyethylene glycol-bound ligand or the polyethylene glycol can be conducted as follows:

A low-molecular compound having an amino group or a carboxyl group can be bound through an amide linkage. In case of a low-molecular compound having an amino group, a carboxyl group is introduced into the polyethylene glycol chain or the polyethylene glycol, and these compounds are condensed by an ordinary method used in the organic synthesis. On the other hand, in a low-molecular compound having a carboxyl group, an amino group is introduced into the polyethylene glycol chain or the polyethylene glycol, and these compounds are condensed by an ordinary method used in the organic synthesis.

Condensation can be conducted by a reaction under dehydro-condensation conditions, specifically at a reaction temperature of 0° C. to room temperature for 1 to 24 hours in a solvent not participating in the reaction (for example, acetonitrile, dimethylformamide, methylene chloride, ethylene chloride) in the presence of an appropriate catalyst (for example, 1-ethyl-3-(3′-dimethylaminopropyl)carbodiimide, N-hydroxysuccinimide, N,N′-dicyclohexylcarbodiimide, 1-hydroxybenzotriazole).

A low-molecular compound having a hydroxyl group or a carboxyl group can be bound through an ester linkage. In a low-molecular compound having a hydroxyl group, a carboxyl group is introduced into the polyethylene glycol chain or the polyethylene glycol, and these compounds are condensed by an ordinary method used in the organic synthesis. On the other hand, in a low-molecular compound having a carboxyl group, such a procedure is unnecessary because a polyethylene glycol has a hydroxyl group.

Condensation can be conducted by a reaction under dehydro-condensation conditions, specifically in a solvent not participating in the reaction (for example, acetonitrile, dimethylformamide, methylene chloride, ethylene chloride) in the presence of an appropriate catalyst (for example, 1-ethyl-3-(3′-dimethylaminopropyl)carbodiimide, N-hydroxysuccinimide, N,N′-dicyclohexylcarbodiimide, 1-hydroxybenzotriazole) at a reaction temperature of 0° C. to room temperature for 1 to 24 hours.

Further, a low-molecular compound having a hydroxyl group can be bound through an ether linkage.

Condensation can be conducted by introducing an active halogen group such as a chloro group, a bromo group and the like and a leaving group such as a tosyl group and the like into the low-molecular compound or the polyethylene glycol chain, and reacting the thus obtained substance with a compound to be bound thereto. Condensation can be conducted by, as required, treatment with a hydride reagent such as sodium hydride, potassium hydride or the like and a reaction in a solvent not participating in the reaction (for example, dimethylformamide, tetrahydrofuran) at a reaction temperature of 0° C. to 100° C. for 1 to 48 hours.

In addition to the foregoing, a low molecular compound and a polyethylene glycol chain of a polyethylene glycol-bound ligand or a polyethylene glycol can be bound by an ordinary method of the organic synthesis depending on the structure of the low-molecular compound using a disulfide linkage, a urethane linkage or the like. In these instances, a functional group for linkage as a spacer may be introduced, as required, for binding.

The medicament of the present invention can be used as a malignant tumor treating agent, an antiviral agent, an antiallergic agent, an immunomodulator, a circulatory function improving agent, an internal secretion function improving agent, an agent for treating or preventing diseases caused by protein abnormal expression or abnormal function, depending on a drug bound to a polyethylene glycol-bound ligand, namely, a physiologically active protein or a low-molecular compound.

The medicament of the present invention can be prepared in any dosage form such as an intravenous or intramuscular injection, a rectal administration agent, an oleaginous suppository, a water-soluble suppository or the like, for example, according to the intended use. These various preparations can be produced by an ordinary method using, as required, an excipient, a bulking agent, a binder, a wetting agent, a disintegrant, a surfactant, a lubricant, a dispersing agent, a buffer agent, a preservative, a solubilizer, an antiseptic, an analgesic, a stabilizer and the like which are commonly used. Examples of the non-toxic additives available include lactose, fructose, glucose, starch, gelatin, magnesium carbonate, synthetic magnesium silicate, talc, magnesium stearate, methylcellulose, carboxymethylcellulose or salts thereof, gum arabic, polyethylene glycol, syrup, vaseline, glycerin, ethanol, propylene glycol, citric acid, sodium chloride, sodium sulfite, sodium phosphate and the like.

When the drug is a protein or a peptide, the administration method of the medicament (medication) in the present invention is preferably parenteral administration such as intravenous administration, subcutaneous administration, intramuscular administration, mucosal administration (application through mucosa) and the like. More preferable is intravenous administration. Further, in case of a low-molecular drug, oral administration is preferable, though its administration method is not limited. Still further, the dose is preferably lower than a saturation binding amount to a receptor for maximizing a target delivery efficiency. In comparison to an ordinary unmodified drug, the dose of the modified drug in the present invention can be decreased (at least ½).

Not only does the dose thereof vary with the drug used, but also it can properly be determined in consideration of the usage, the age, the sex and the degree of progression of the disease of the patient, and the like. When the medicament in the present invention, namely, the medicament in which the drug is bound to the polyethylene glycol chain of the polyethylene glycol-bound ligand, for example, polyethylene glycol-bound (Gal)3-modified IFNα, polyethylene glycol-modified IFNα and (Gal)3-modified IFNα is each intravenously administered to mice to measure the in-vivo behavior, the compound in the present invention, in comparison to the product modified with only the ligand ((Gal)3-modified IFNα), suppresses disappearance in the initial stage of administration by avoiding glomerular filtration in the kidney and also avoids disappearance in other organs in circulating blood to greatly improve a retention thereof in the liver. Moreover, with respect to the change in concentration in other organs, the compound in the present invention is retained at a lower concentration than the product modified with polyethylene glycol only (polyethylene glycol-modified IFNα).

Thus, the medicament is accumulated and retained at a high concentration in a target tissue, and is retained at a low concentration in other organs. It is therefore suggested that a therapeutic index of IFNα can be increased.

The dose of the medicament in the present invention can be decreased down to between ½ and 1/9 of the dose of the medicament with the unmodified product owing to the target accumulation and the long-term retention properties, and thus the present invention allows therapy with side effects reduced. Further, since the introduction of the polyethylene glycol-bound ligand to the drug is conducted by one step, the polyethylene glycol-bound ligand modified drug can be obtained at a high recovery rate by the simple production step. Still further, since the medicament of the present invention is a product modified with only one molecule of the polyethylene glycol-bound ligand, it is possible to easily prove the same by a peptide map or the like and ensure fixed qualities as a medicament.

Having generally described this invention, a further understanding can be obtained by reference to certain specific examples, which are provided herein for purposes of illustration only, and are not intended to be limiting unless otherwise specified.

### EXAMPLES

### Example 1

### Synthesis of a Polyethylene Glycol-Bound Ligand

A polyethylene glycol derivative is synthesized in which a Boc-protected alkylamine (HOOC—(CH₂)₅—NHBoc) is introduced into one end of polyethylene glycol having carboxyl groups in both ends (average weight-average molecular weight 9,000 Da, HOOC—CH₂—(OCH₂CH₂)ₙ—O—CH₂—COOH) to become a substrate of a transglutaminase. A glutamic acid-tribranched galactose ligand derivative obtained by removing a terminal alkylamine from (Gal)₃ formed by the method described in official gazette of WO 96/06181 and O-acetylating a hydroxyl group of galactose is introduced into the carboxyl group at the opposite end of this Boc alkylamine-introduced polyethylene glycol by an active ester method. The ligand, the terminal Boc group of the alkylamine-introduced polyethylene glycol and the OAc group are removed in order to obtain a polyethylene glycol-bound (Gal)₃.

### Example 2

### Synthesis of a Medicament (Medication) in Which a Drug is Bound to a Polyethylene Glycol Chain of a Polyethylene Glycol-Bound Ligand

The polyethylene glycol-bound (Gal)₃ obtained in Example 1 is reacted with human interferon α (IFNα) in the presence of a microbial transglutaminase by the method described in official gazette of WO 96/10089 to obtain an IFNα modified with polyethylene glycol-bound (Gal)₃ at a high recovery rate.

### Example 3

### Synthesis of a Polyethylene Glycol with an Alkylamine Introduced in One End

Synthesis of a polyethylene glycol with an alkylamine introduced in one end was conducted by the method described by Sato et al. (Biochemistry, 35(40), 13072 (1996)). Dry DMF (55 ml) was added to 5.00 g of dicarboxylic acid-type polyethylene glycol (made by Nippon Oils and Fats Co., Ltd., average weight-average molecular weight 12,000 Da) [compound (1), page 20], and the mixture was slightly heated for dissolution. The solution was cooled to 25° C., and 101 mg (0.75 mmol) of 1-hydroxybenzotriazole (HOBt) and 144 mg (0.75 mmol) of EDCI (1-ethyl-3-(3-dimethylaminopropyl)carbodiimide) were added thereto.

After the mixture was stirred at the same temperature for 30 minutes, a DMF (5 ml) solution of 152 mg (0.75 mmol) of t-butyl N-(5-aminoamyl)carbamate [compound (2), page 20] was added dropwise thereto. The reaction solution was heated at 35° C., and the reaction was conducted for 16 hours. HOBt (101 mg, 0.75 mmol) and 144 mg (0.75 mmol) of EDCI were added to this mixture. After the reaction was conducted for 11 more hours, 101 mg (0.75 mmol) of HOBt and 144 mg (0.75 mmol) of EDCI were then added. After the addition thereof, the reaction was continued for 11 more hours, and the reaction solution was then concentrated to obtain 8.37 g of a crude product.

The crude product was column purified (silica gel 300 g, eluent chloroform/methanol=95/5→92/8), and divided into a fraction of 0.83 g and a fraction of 1.46 g. The 0.83 g fraction was further column purified (silica gel 300 g, eluent chloroform/methanol=95/5→50/50) to obtain 500 mg of a purified product (purified product 1). The 1.46 g fraction was further column purified (silica gel 300 g, eluent chloroform/methanol=95/5→50/50) to obtain 1,058 mg of a purified product (purified product 2). The structures of the purified products were confirmed by ¹H-NMR such that only a half amount of a signal ascribable to an ethyl group adjacent to a terminal carboxyl group was shifted and a main signal ascribable to an ethylene glycol group in the PEG skeleton was retained.

### Example 4

### Synthesis of Compound (5)

Trifluoroacetic acid (6.55 ml) was added dropwise to a dichloromethane (10 ml) solution of compound (4) [see page 20] (1.41 g, 0.81 mmol), which was synthesized by the method described in the official gazette of WO 01/02851 in an argon stream at 0° C. for 30 minutes. After completion of the dropwise addition of trifluoroacetic acid, the reaction temperature was increased to 20° C., and the reaction was conducted for 30 minutes. The reaction solution was subsequently concentrated, and thrice subjected to azeotropic distillation with ethanol to remove as much trifluoroacetic acid as possible. The resulting crude product was column purified (silica gel 80 g, eluent chloroform→chloroform/methanol=5/1) to obtain 1.35 g of a column purification product. This product was column purified again (silica gel 50 g, eluent chloroform→chloroform/methanol=8/1) to obtain 457 mg of the compound (5) (yield 34%). The structure of the purified product was confirmed by ¹H-NMR such that a terminal Boc group was removed from the compound (4) [see page 20]. Further, its purity was identified by a thin layer chromatography (TLC).

### Example 5

Synthesis of (Gal(OAc)₄)₃-PEG(Boc)

In an argon stream, 670 g of compound (3), 51.4 mg (268 mmol) of EDCI and 35 ml of dry DMF were added, and 36.2 mg (268 mmol) of HOBt was further added thereto. To this solution, a dry DMF (30 ml) solution of 220 mg (134 mmol) of compound (5) was added dropwise. The temperature of the reaction solution was increased, and the reaction was conducted at 33° C. for 14 hours. The reaction solution was concentrated to obtain 1.08 g of a crude product, which was column purified (silica gel 100 g, eluent chloroform/methanol=95/5→93/7) to obtain 697 mg of (Gal(OAc)₄)₃-PEG(Boc). By this same method, 619 mg and 109 mg of (Gal(OAc)₄)₃-PEG(Boc) were obtained respectively from 610 mg and 102 mg of compound (3) [see page 20]. The structures of the purified products were confirmed from an identification of terminal Boc group and OAc group as a Gal protective group and further from a retention of a main signal ascribable to an ethylene glycol group in a PEG skeleton moiety as measured by ¹H-NMR (solvent CDCl₃). Further, the purities thereof were confirmed by TLC.

### Example 6

### Synthesis of (Gal(OAc)₄)₃-PEG

A dichloromethane (20 ml) solution of 20 ml of trifluoroacetic acid was added dropwise to a dry dichloromethane (100 ml) solution of 1.18 g of the (Gal(OAc)₄)₃-PEG(Boc) in an argon stream at 1° C. for 1.2 hours. The temperature of the reaction solution was increased to 14° C. over a period of 1 hour for concentration. One hundred milliliters (100 ml) of dry ethanol was added to assist concentration, and a procedure for removing trifluoroacetic acid was repeated twice. The resulting residue was dissolved in 10 ml of methanol, and the solution was neutralized with a 28% NaOCH₃ methanol solution diluted 10-fold. The reaction solution was concentrated, and the resulting crude product was purified on a column (silica gel 100 g, eluent chloroform/methanol=95/5→1/1) to obtain 606 mg of a purified product. This product was combined with 100 mg of a preliminary examination product, which was also purified on the silica gel column once, and the combination was repeatedly subjected to a column purification on the same scale four times to obtain 295 mg of (Gal(OAc)₄)₃-PEG. The structure of the purified product was confirmed by removal of the Boc group from the (Gal(OAc)₄)₃-PEG(Boc) through ¹H-NMR. Further, its purity was confirmed by TLC.

### Example 7

### Synthesis of (Gal)₃(6)-PEG

A 28% NaOCH₃ methanol solution (23 mg) was diluted with 15 ml of methanol and then added dropwise to a methanol solution of 290 mg of the (Gal(OAc)₄)₃-PEG at 3° C. After the dropwise addition, a reaction was conducted at 15 to 21° C. for 3.5 hours. Since a spot in the same site as that of the starting material was not decreased in the tracing of the reaction by TLC, 22 mg of a 28% NaOCH₃ methanol solution was diluted with 15 ml of methanol, and then added thereto dropwise. After this dropwise addition, the reaction was conducted at 20 to 21° C. for 1.5 hours. The reaction solution was cooled, and Dowex 50W×8 cleaned with methanol was added in small amounts to neutralize the solution. The reaction solution was subsequently filtered, and the filtrate concentrated to dryness resulting in 250 mg of (Gal)₃(6)-PEG. The structure of the purified product was identified by confirming separation of the OAc group from the (Gal(OAc)₄)₃-PEG through ¹H-NMR. Further, the purity was confirmed by TLC.

### Example 8

### Preparation of (Gal)₃(6)-PEG-IFNα

One hundred micrograms (100 μg)of freeze-dried human IFNα(2b) (obtained from Seikagaku Kogyo K.K.) was dissolved in 744 μl of a 200 mM tris-hydrochloride buffer solution (pH 7.5), and 57.3 mg of (Gal)₃(6)-PEG obtained in Example 7 was added thereto. Approximately 0.7 U of a microbial transglutaminase was added to the reaction solution, and the mixture was incubated overnight at room temperature. The reaction solution was subjected to a SDS-PAGE (homogenious 20 (made by Pharmacia)) to confirm the disappearance of unreacted IFNα. Then, unreacted (Gal)₃-PEG and M-TG were removed using a “SEP-PAK” column (made by Millipore). The resulting fraction obtained was concentrated with Speed Vac, and neutralized with PBS (+). Approximately 4.4 μg of (Gal)₃(6)-PEG-IFNα was obtained in this reaction.

The reactions described in Examples 3 to 8 are shown below using reaction formulas.

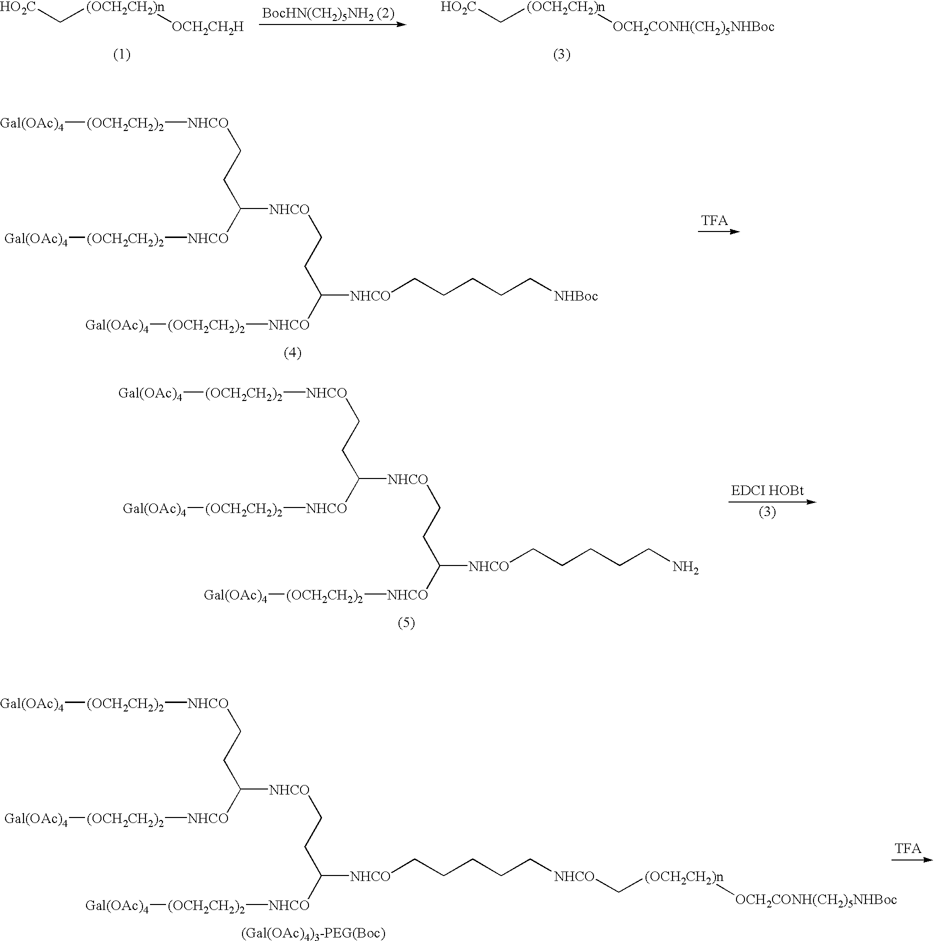

### Example 9

### In-Vivo Behavior of (Gal)₃(6)-PEG-IFNα

To the tail of individual 6-week-old male mice (C57BL/6, CRJ) were intravenously administered 19.9 μg/kg, calculated as IFNα, of (Gal)₃(6)-PEG-IFNα, PEG12-IFNα (PEG one molecule modified product having an average weight-average molecular weight of 12 kDa and obtained by chemically modifying one end methoxy PEG carboxylic acid made by Nippon Oils and Fats Co., Ltd. through an active ester method and then collecting only a one molecule modified product through a reversed phase HPLC (Sato et al., Bioconjugate Chem., 11(4), 502 (2000)), (Gal)₃(6)-IFNα (prepared according to the method described in the official gazette of WO 01/02851) and unmodified IFNα, respectively.

After the lapse of a fixed time (3 minutes or 60 minutes), blood sampling and sampling of the liver were conducted. Plasma sampling was performed by a standard procedure, and the concentration in the specimen was measured by ELISA. The liver was homogenized by adding a homogenate buffer (MEM, 5% FBS, pH 7.2 to 7.4) in a 9-fold 5 amount of the weight of the organ, and centrifuged at 3,000 rpm for 10 minutes.

Subsequently, a supernatant was diluted with the same homogenate buffer, and a concentration in the specimen was measured by ELISA. Incidentally, ELISA was conducted using a Human ELISA IFNα kit (made by ENDOGEN).

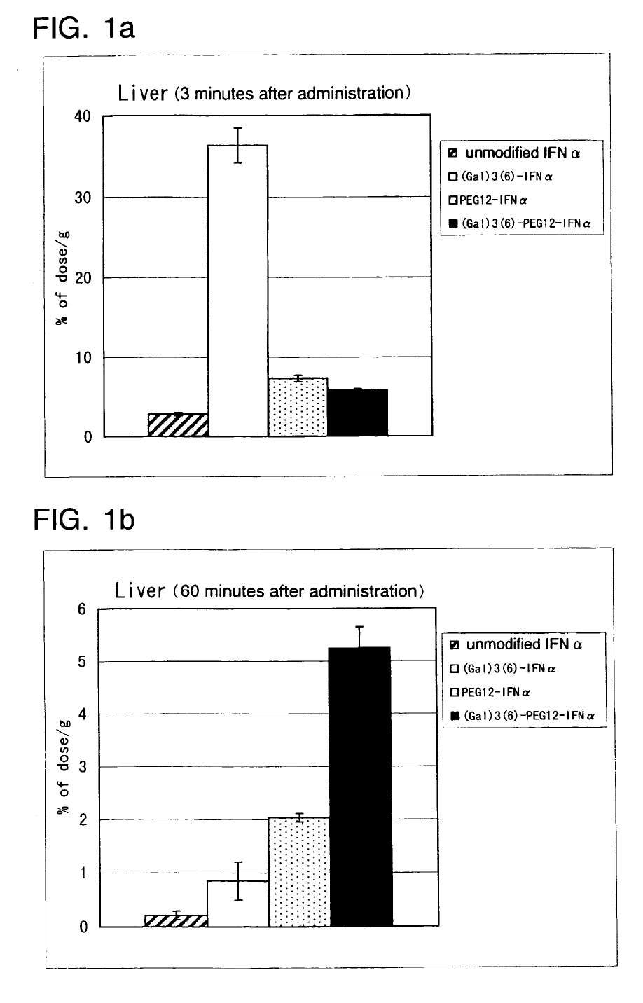

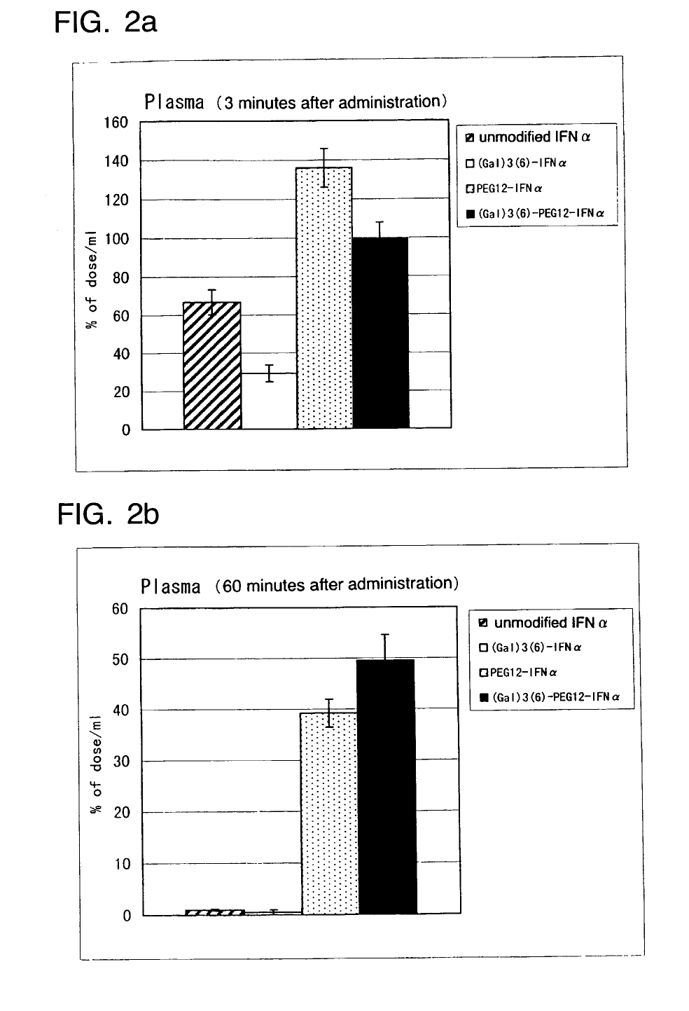

Consequently, as shown in FIGS. 1 and 2, in the initial stage of the administration, (Gal)₃(6)-PEG-IFNα, like PEG12-IFNα, exhibited quite a high concentration in plasma (refer to FIG. 2a) and a low concentration of accumulation in the liver in comparison to (Gal)₃(6)-IFNα (refer to FIG. 1a). However, after 1 hour from the administration, the concentration of (Gal)₃(6)-PEG-IFNα in the liver was retained highest (refer to FIG. 1b), and it was approximately 2.6 times as high as that of PEG12-IFNα and approximately 6.2 times as high as (Gal)₃(6)-IFNα. This means that even after the lapse of a long time the concentration in the liver is retained high owing to the liver accumulation ability by the ligand and the retention effect by PEG. The foregoing results proved that an accumulation ability in the liver and also a circulating property in the liver were imparted by providing the (Gal)₃(6) ligand as a liver recognition element and also PEG, and the usefulness of the novel ligand was clarified.

Numerous modifications and variations on the present invention are possible in light of the above teachings. It is, therefore, to be understood that within the scope of the accompanying claims, the invention may be practiced otherwise than as specifically described herein.

The present application is a continuation of and claims priority to PCT/JPO1/02604, filed on Mar. 28, 2001, which is hereby incorporated by reference in its entirety. In addition, the present application claims priority to JP 2000-93775, filed on Mar. 30, 2000, which is also incorporated by reference in its entirety.

What we claim is:

1. A polyethylene glycol-bound ligand comprising:
a polyethylene glycol chain bound to
a ligand having a binding affinity to a cell surface receptor or cell surface protein,
wherein said polyethylene-bound ligand avoids and/or inhibits cellular internalization of said polyethylene-bound ligand.

2. The polyethylene glycol-bound ligand of claim 1, wherein said surface receptor or cell surface protein is an antigen.

3. The polyethylene glycol-bound ligand of claim 1, wherein said ligand binds to a cell surface protein or glycoprotein.

4. The polyethylene glycol-bound ligand of claim 1, wherein said binds to a cell surface receptor.

5. The polyethylene glycol-bound ligand of claim 1, wherein said ligand binds to a cell surface receptor that is not a protein.

6. The polyethylene glycol-bound ligand of claim 1, wherein the ligand binds to a specific receptor present on the cell membrane of a target cell.

7. The polyethylene glycol-bound ligand according to claim 1,
wherein a ratio of a dissociation rate constant and an internalization rate constant of the ligand through the specific receptor is 1 or more.

8. The polyethylene glycol-bound ligand according to claim 7, wherein a dissociation constant of the ligand to the specific receptor is 10 mM or less.

9. The polyethylene glycol-bound ligand of claim 7, wherein the internalization rate constant of the ligand to the specific receptor is less than 0.1 min⁻¹.

10. The polyethylene glycol-bound of claim 1, wherein said ligand binds an asialoglycoprotein receptor (ASGP-R).

11. The polyethylene glycol-bound ligand of claim 1, wherein the target cell or tissue is a liver cell or tissue.

12. The polyethylene glycol-bound ligand of claim 1, wherein the cell or target tissue is a kidney cell or tissue.

13. The polyethylene glycol-bound ligand of claim 1, wherein the target cell or tissue is a bone marrow cell or tissue.

14. The polyethylene glycol-bound ligand of claim 1, wherein the target cell or tissue is a pancreas cell or tissue.

15. The polyethylene glycol-bound ligand of claim 1, wherein said ligand binds a receptor selected from the group consisting of a mannose receptor, an epidermal cell growth factor (EGF) receptor, an insulin receptor, a transferrin receptor, and a folate receptor.

16. A medicament comprising a drug bound to the polyethylene glycol chain of the polyethylene glycol-bound ligand according to claim 1.

17. The medicament of claim 16, wherein the drug is a physiologically active protein or a physiologically active peptide.

18. The medicament of claim 17, wherein the physiologically active protein is one or more protein selected from the group consisting of an immunoglobulin, a blood coagulation factor, an interleukin, an interferon, a tumor necrosis factor, a hepatocyte growth factor (HGF), an epidermal cell growth factor, superoxide disumtase (SOD), catalase or thioredoxin.

19. The medicament of claim 17, wherein the physiologically active protein comprises a glutamine residue and has a weight-average molecular weight of 1×10³ to 2×10⁵.

20. The medicament of claim 17, wherein the physiologically active protein is interferon α, interferon β or interleukin-2.

21. The medicament of claim 16, wherein the drug is selected from the group consisting of adriamycin, mitomycin C, methotrexate, azidothymidine, adenine arabinoside, and ribavirin

22. The medicament of claim 16, wherein the end of the polyethylene glycol chain of the polyethylene glycol-bound ligand is an amino group and which can be produced by reacting the amino group with a physiologically active protein in the presence of a transglutaminase to form an amide linkage.

23. A pharmaceutical composition comprising the medicament of claim 16 and a pharmaceutically acceptable carrier.

24. The medicament of claim 16, wherein the drug is a physiologically active protein or a physiologically active peptide.

25. The medicament of claim 24, wherein the physiologically active protein is one or more protein selected from the group consisting of an immunoglobulin, a blood coagulation factor, an interleukin, an interferon, a tumor necrosis factor, a hepatocyte growth factor (HGF), an epidermal cell growth factor, superoxide disumtase (SOD), catalase or thioredoxin.

26. The medicament of claim 24, wherein the physiologically active protein comprises a glutamine residue and has a weight-average molecular weight of 1×10³ to 2×

27. The medicament of claim 24, wherein the physiologically active protein is interferon α, interferon β or interleukin-2.

28. The medicament of claim 23, wherein the drug is selected from the group consisting of adriamycin, mitomycin C, methotrexate, azidothymidine, adenine arabinoside, and ribavirin

29. The medicament of claim 23, wherein the end of the polyethylene glycol chain of the polyethylene glycol-bound ligand is an amino group and which can be produced by reacting the amino group with a physiologically active protein in the presence of a transglutaminase to form an amide linkage.
